(19) United States
(12) Patent Application Publication
(10) Pub. No.: US 2008/0220078 A1
(43) Pub. Date: Sep. 11, 2008

(54) Pharmaceutical Formulations

(75) Inventors: David Alexander Vodden Morton, Wiltshire (GB); Robert Price, Wiltshire (GB); Philippe Begat, Wiltshire (GB)

Correspondence Address: Davidson, Davidson & Kappel, LLC, 485 7th Avenue, 14th Floor, New York, NY, 10018, US

(73) Assignee: Vectura Limited, Chipperharm, Wilshire (GB)

(21) Appl. No.: 11/791,670

(22) PCT Filed: Nov. 30, 2005
(86) PCT No.: PCT/GB2005/050220

§ 371 Date: Dec. 26, 2007

(30) Foreign Application Priority Data
Nov. 30, 2004 (GB) 0426301.8

(51) Int. Cl. A61K 9/14; A61K 9/12; A61K 39/395

(52) U.S. Cl. 424/490; 424/43; 424/45; 424/130.1

(57) ABSTRACT

A pharmaceutical formulation for delivery in aerosol or spray form, comprising a liquefied propellant gas, a solid particulate pharmaceutically active agent and a dispersing agent, wherein the dispersing agent is fused to the surface of particles of the pharmaceutically active agent.

The present invention relates to formulations for delivery in aerosol form, particularly pharmaceutical aerosol formulations for pulmonary, nasal, buccal or topical administration, to methods of manufacturing such formulations, and to products, preferably pharmaceutical products, that comprise such formulations.

Spray and aerosol formulations have been employed for many years to administer pharmaceutically active agents both topically and for systemic absorption. For example, pressurised metered dose inhalers (pMDI), capable of administering a metered dose of an aerosol of a pharmaceutically active agent to the lungs, have been in use for many decades, particularly in the topical treatment asthmatic conditions. Such devices are well known and examples are disclosed in WO92/11190, U.S. Pat. No. 4,819,834 and U.S. Pat. No. 4,407,481.

The formulations employed in pMDI and other like aerosol and spray devices generally comprise a pharmaceutically active agent dissolved or suspended in a liquefied propellant gas. Chlorofluorocarbons (CFC), which have traditionally been employed as propellants in such formulations, were implicated in the depletion of the ozone layer over fifteen years ago and their use, therefore, is being phased out. It was determined, somewhat over a decade ago, that certain hydrofluorocarbons (HFA), that are both of low toxicity and of suitable vapour pressure for use as aerosol propellants, are significantly less harmful to the ozone layer than CFCs. Among these HFAs, 1,1,1,2-tetrafluoroethane (HFA-134a) and 1,1,1,2,3,3,3-heptafluoropropane (HFA-227) have been proposed as suitable propellants for use in pharmaceutical aerosol formulations. See, for example, EP 0 372 777, EP 0 499 344 and EP 0 553 298.

However, despite the fact that these latter proposals were first made more than 10 years ago, very few pharmaceutical aerosol products, which employ either HFA-134a or HFA-227 as propellants, have appeared in the market place.

As noted above, the pharmaceutically active agents present in formulations used in pMDI, and other like propellant driven devices, are either dissolved or suspended in a liquefied propellant gas. Most pharmaceutically active agents are not sufficiently soluble in pure propellants, either HFAs or CFCs, for simple two component formulations of active agent and propellant to be practical. Although, through the incorporation of a co-solvent such as ethanol, many active agents can be dissolved in the resulting formulation, formulations in which the active agent, in a micronised or particulate form, is suspended in the propellant are generally preferred and more common. There are several reasons for this. It is important to control the size of the particles or droplets in the aerosol spray produced by a pMDI, or like device. For example, if the particles or droplets are to penetrate deep into the lungs, they should have a mass median aerodynamic diameter (MMAD) of less than 10 μm. Conversely, if the spray is for buccal or nasal delivery, the particles or droplets must have an MMAD of significantly greater than 10 μm, in order to prevent them from entering the lungs. Controlling the size of the particles in an aerosol spray produced from a purely liquid formulation is more difficult than it is with a formulation comprising a suspended solid particulate pharmaceutically active agent. In the former case, many environmentally influenced factors, such as solvent evaporation rates, will have an effect on particle size, whereas the size of the particles produced by a suspension formulation is determined largely by the size of the active agent particles employed in its preparation, and this is a parameter that can be effectively controlled.

A second, but important, reason for suspension formulations being preferred, is that many pharmaceutically active agents are chemically more stable as solids than they are when in solution. For example, most pharmaceutically active compounds are much more susceptible to degradation by acid or alkali when in solution than they are when solid. It is also simply impossible to render many pharmaceutically active agents sufficiently soluble in a pharmaceutically acceptable propellant system, for a solution formulation to be a realistic option for them.

Dispersing agents, such as surfactants, are commonly employed in suspension aerosol formulations in order to ensure that the particles of pharmaceutically active agent can be dispersed within the propellant system without an undue degree of agitation and remain so dispersed for a sufficiently long period of time for the effective operation of the pMDI to be ensured. Surfactants can also provide useful lubrication to the metering valve's mechanism. However, one of the problems which has arisen in the development of HFA based suspension formulations for use in pMDI and like devices, is that many of the surfactants commonly employed as dispersing agents in CFC based formulations are substantially insoluble in HFA-134a and HFA-227 and, thus, are substantially ineffective in simple formulations based on these latter two propellants, or other HFA propellants.

One solution to this problem, which was proposed in EP 0 372 777, is to incorporate a co-solvent, such as ethanol, having a greater polarity than the HFA propellant in the formulation, in order to dissolve the surfactant or other dispersing agent. Whilst the presence of such a co-solvent allows most dispersing agents to be dissolved in HFA based formulations, it will also cause certain pharmaceutically active agents to dissolve, at least partially, in the co-solvent/propellant system. This phenomenon is especially disadvantageous in formulations for delivery into the lungs because, over time, it causes the particles of active agent in the formulation to grow, possibly to a size in excess of that generally considered to be acceptable for inhalation, i.e., to have a MMAD of greater than 10 μm. Further disadvantages associated with the use of ethanol as a co-solvent include its potential toxicity, its capacity to increase a formulation's propensity to absorb water and the fact that many patients dislike the taste its presence can impart to a formulation.

Another method for incorporating a surfactant or dispersing agent, which has been proposed in the literature, is to coat the particles of pharmaceutically active agent with the surfactant or dispersing agent before they are mixed with the propellant and to suspend the coated particles in the HFA propellant, without using any co-solvent. In detail, it was proposed to coat the active agent using a process involving the steps of dissolving the surfactant in a solvent in which the pharmaceutically active agent is substantially insoluble, mixing a quantity of the pharmaceutically active agent, in micronised form, into the surfactant solution and isolating particles of surfactant coated active agent either by filtration and drying, or by removal of the solvent by evaporation. Although the literature suggests (see WO92/08447 and WO91/04011) that formulations prepared in this manner are effective, in the sense that they allow stable dispersions of powdered active agent to be formed in the HFA propellant, it has so far not proven possible, in practice, to manufacture useful formulations in this way. For example, it is difficult to achieve a uniform coating using techniques of this nature because the manner in which the dispersing agent precipitates from the evaporating solvent can be unpredictable.

A further technique, which has recently been proposed, is to suspend a powdered mixture consisting of particles of a calcium, magnesium or zinc salt of palmitic or stearic acid and particles of pharmaceutically active agent in the propellant. Once again, although the literature suggests that formulations prepared in this manner are effective (see US 2004/0101483), none have yet to be commercialized.

It has now been found, surprisingly, that if a dispersing agent is fused onto the surface of solid particles of pharmaceutically active agent by a coating technique that comprises bringing solid dispersing agent into contact with solid particles of the active agent, and applying mechanical energy to the contacting dispersing agent and particles of active agent, the resulting composite or hybrid particles are sufficiently readily dispersible within HFA propellant systems, for such dispersions to provide the basis for commercially viable formulations that do not include a co-solvent such as ethanol. Examples of coating techniques that can apply the energy required to cause fusion include the dry techniques described in R. Pfeffer et al. “Synthesis of engineered particulates with tailored properties using dry particle coating”, Powder Technology 117 (2001) 40-67. These include, in addition to processes using the MechanoFusion® machine, those employing the Hybidizer® machine, the Theta Composer®, magnetically assisted impaction processes and rotating fluidised bed coaters. Other methods that can be used include dry ball milling and like processes.

According to a first aspect of the present invention, therefore, there is provided a pharmaceutical formulation for delivery in aerosol or spray form, comprising a liquefied propellant gas, a solid particulate pharmaceutically active agent and a dispersing agent, wherein the dispersing agent is fused to the surface of particles of the pharmaceutically active agent. The dispersing agent and pharmaceutically active agent are preferably in the form of solid composite particles. Said particles can be suspended or are suspendable in the liquefied propellant gas and each such particle, preferably, comprises a particle of the pharmaceutically active agent at least partially coated with the dispersing agent, that is or can be suspended in the liquefied propellant gas.

The dispersing agent is preferably in the form of a coating on the surfaces of the particles of pharmaceutically active agent. The coating can be a discontinuous coating and can be in the form of particles of dispersing agent fused to the surfaces of the particles of pharmaceutically active agent.

In further embodiments, the dispersing agent forms an at least partial coating or shell around a plurality of particles of active agent. Preferably, at least 50, 70, 80, 90 or 95% of the surface area of the active agent is coated or covered with dispersing agent.

In a second aspect, the present invention provides a method for preparing a pharmaceutical formulation in accordance with the first aspect of the invention, comprising fusing the dispersing agent to the surface of particles of a solid particulate pharmaceutically active agent and admixing the solid particulate pharmaceutically active agent and dispersing agent with a liquefied propellant gas. The liquefied propellant gas can be admixed with the dispersing agent and particulate pharmaceutically active agent before, during and/or after the dispersing agent is fused to the particulate pharmaceutically active agent. However, it is preferred for at least some, preferably a majority and more preferably substantially all of the propellant to be added after the dispersing agent has been fused to the pharmaceutically active agent.

Preferably, the dispersing agent is fused to the surface of solid particles of pharmaceutically active agent by a method comprising bringing solid dispersing agent into contact with the particles of pharmaceutically active agent, and applying sufficient mechanical energy to contacting dispersing agent and particles of pharmaceutically active agent to cause fusion between them.

In preferred embodiments of the second aspect of the invention, the mechanical energy is applied to a, preferably dry, mixture of dispersing agent and active agent particles. In many preferred embodiments of the second aspect of the invention, the mechanical energy is provided in the form of simultaneous compression and sheer forces applied to the contacting dispersing agent and active agent particles.

The dispersing agent is preferably softer and/or more malleable than the pharmaceutically active agent within the temperature range at which the method in accordance with the second aspect of the invention is carried out. The dispersing agent can be softer and/or more malleable than the pharmaceutically active agent at a temperature in the range of 20-80° C. It is preferred for the dispersing agent to be sufficiently soft and malleable, relative to the pharmaceutically active agent, such that it can be deformed, smeared or spread across and fused to the surfaces of the pharmaceutically active agent particles by the application of mechanical energy to contacting dispersing agent and particles of pharmaceutically active agent. The dispersing agent can be lamellar in nature, comprising alternate hydrophobic and hydrophilic elements that, when subjected to sufficient mechanical energy, shear along the planes in which they are stacked.

In preferred embodiments of the second aspect of the invention, sufficient mechanical energy is applied to contacting particles of dispersing agent and pharmaceutically active agent to cause the dispersing agent particles to soften and/or distort such that the dispersing agent spreads across to at least partially coat the surfaces of the pharmaceutically active agent particles.

Preferably and unlike in some previously known arrangements, the particles of dispersing agent are smaller than the particles of pharmaceutically active agent and each of the composite particles can comprise, or consist or consist essentially of a plurality of dispersing agent particles fused to the surface of a particle of pharmaceutically active agent.

It is preferred for the particles of pharmaceutically active agent employed in methods in accordance with the second aspect of the invention to be of substantially the same size (MMAD) as the composite particles produced by the method. In this regard, the method in accordance with the second aspect of the invention can cause some degree of reduction in the size of the active agent particles. However, the relative sizes of the active agent and dispersing agent particles employed in the inventive method is such that the fusing of particles of dispersing agent to the active agent particles does not substantially alter the size, or MMAD of the latter. For example, whilst the particles of active agent can range in size between 0.1 and 100 μm, the MMAD of the dispersing agent particles, typically, will not exceed 1 μm. The ratio between the MMAD of the dispersing agent particles to the MMAD of the pharmaceutically active agent particles employed in the method in accordance with a second aspect of the invention, typically, will be less than 1:10, preferably 1:20 or less and, more preferably, 1:100 or less.

The dispersing agent particles can, in embodiments, be larger before the mechanical energy is applied to them. In these circumstances, the mechanical energy is sufficient to reduce the dispersing agent particles in size so that the ratio of their size to that of the particles of active agent falls within at least one of these latter preferred ranges. It is also preferred that the applied mechanical energy does not cause any significant change to the size of the particles of the pharmaceutically active agent.

By carefully selecting the dispersing agent or agents from amongst materials that have the above discussed properties, it is possible to provide solid composite particles in accordance with the first aspect of the invention capable of providing a controlled or delayed release of the active agent in the lung and/or that have improved resistance to moisture ingress. By the same means, it is also possible to prevent or reduce the tendency of chemical interaction between particles of different active agents, or between particles and components of the pMDI device.

In preferred embodiments of the first aspect of the invention, the formulation has been prepared, or is preparable by a method in accordance with the second aspect of the invention.

In preferred embodiments, the bond between the dispersing agent and the active agent is physical and preferably involves physisorption of the dispersing agent by the pharmaceutically active agent and/or vice versa.

A further advantage of the present invention in all of its aspects is that it allows many of the surfactants that have been used in CFC based formulations, all of which have been shown to be safe over many years of use, to be used as dispersing agents with non-CFC based propellant systems. In preferred embodiments of the invention the dispersing agent comprises such a surfactant.

Preferably, the dispersing agent is substantially insoluble in the liquefied propellant gas. In embodiments, the dispersing agent reduces the surface free energy of the particles of pharmaceutically active agent.

In further preferred embodiments, the dispersing agent has a molecular weight of at least about 5500 or 6000.

In preferred embodiments the amount of dispersing agent in the composite particles will not be more than 60% by weight. Preferably, the composite particles should comprise 40 to 0.25%, more preferably 30 to 0.5%, and even more preferably 20 to 2% by weight dispersing agent. In further embodiments, the composite particles can comprise 2 to 10% or 3 to 8% by weight dispersing agent.

Advantageously, the dispersing agent is an anti-adherent material and will decrease the cohesion between the composite particles and between the composite particles and the components of a pMDI device, when the formulation is contained within such a device. The dispersing agent can also be an anti-friction agent (glidant). The dispersing agent may include a combination of one or more materials (e.g. surfactants) and it is preferred that the dispersing agent is or includes a naturally occurring animal or plant substance. The reduced tendency of the particles to bond strongly, either to each other or to the device itself leads to improvements in dose to dose consistency because it reduces the variation in the quantity of active agent containing composite particles metered out for each dose.

Preferably, the dispersing agent comprises, or consists or consists essentially of one or more amino acid, amino acid derivative, peptide and/or peptide derivative. Of these, amino acids are preferred. Thus, in particular, the dispersing agent can comprise one or more of the following amino acids: leucine, isoleucine, lysine, valine, methionine and phenylalanine. The dispersing agent can be salt or a derivative of an amino acid, for example it can be or include aspartame or acesulfame K. The dispersing agent can consist essentially of an amino acid, preferably leucine and, more preferably L-leucine. The D- and DL-forms, however, can be used.

The dispersing agent can comprise, or consist or consist essentially of one or more water soluble substances, as such substances are generally readily absorbed by the body should they reach the lower lung. The dispersing agent may include dipolar ions which may be zwitterions. It can also be advantageous to include a spreading agent as a dispersing agent, to assist with the dispersal of the composition in the lungs. Suitable spreading agents include surfactants such as known lung surfactants (e.g. ALEC™) which comprise phospholipids, for example, mixtures of DPPC (dipalmitoyl phosphatidylcholine (and PG phosphatidylglycerol). Other suitable surfactants include, for example, dipalmitoyl phosphatidylethanolamine (DPPE), dipalmitoyl phosphatidylinositol (DPPI).

The dispersing agent can comprise, or consist or consist essentially of a metal stearate or palmitate. It can also comprise a derivative of such a salt or acid, such as, for example, sodium stearyl fumarate or sodium stearyl lactylate. Preferred metal stearates and palmitates include zinc, magnesium, calcium, sodium and lithium stearates and palmitates, with the stearates being more preferred. Of the latter, magnesium stearate is the most preferred. When used, magnesium stearate is preferably in the form of vegetable magnesium stearate, but it can be in any commercially available form, which may be of vegetable or animal origin, and may also contain other fatty acid components, such as palmitates or oleates.

The dispersing agent can comprise, or consist or consist essentially of one or more surface active agent, in particular, a material that is surface active in the solid state, which may also be water soluble, such as lecithin, particularly soya lecithin, or substantially water insoluble, for example, solid state fatty acids such oleic acid, lauric acid, palmitic acid, stearic acid, erucic acid, behenic acid, and derivatives (such as esters and salts) thereof, such as glyceryl behenate. Specific examples of such materials include phosphatidylcholines, phosphatidylethanolamines, phosphatidylglycerols and other examples of natural and synthetic lung surfactants, lauric acid and its salts, for example, sodium lauryl sulphate, magnesium lauryl suphate, triglycerides such as Dynsan 118 and Cutina HR and sugar esters in general. Alternatively, the dispersing agent can be cholesterol.

Further examples of materials that can be employed as dispersing agents include sodium benzoate, hydrogenated oils which are solid at room temperature, talc, titanium dioxide, aluminium dioxide, silicon dioxide and starch. Further useful dispersing agents include film forming agents, fatty acids and their derivatives, as well as lipids and lipid like materials.

It is preferred for the dispersing agent to comprise, or consist or consist essentially of one or more of the following: amino acids, lecithins, phospholipids, sodium stearyl fumarate, glyceryl behenate and metal stearates (especially magnesium stearate). However, it is most preferred for the dispersing agent to comprise or consist or consist essentially of a phospholipid, preferably a lecithin, or a metal stearate, preferably magnesium stearate.

The particles of pharmaceutically active agent preferably comprise one or more pharmaceutically active compound and can consist or consist essentially of one or more pharmaceutically active compound. The pharmaceutically active agent can be suitable for therapeutic and/or prophylactic use. The pharmaceutically active agent can comprise any such agent that is administrable as a spray or aerosol formulation either topically, or for systemic absorption. Suitable pharmaceutically active agents for inclusion in formulations in accordance with the invention include those that can be administered by a pulmonary, nasal or buccal route. Amongst such agents include those typically employed in the treatment of respiratory conditions. These include β₂-agonists, such a terbutaline, salbutamol, salmeterol and formoterol, antimuscarinics, such as ipratropium bromide, tiotropium bromide, steroids, such as beclomethasone and fluticasone, cromones, such as sodium cromoglycate and nedocromyl and their physiologically acceptable salts and esters, such as salbutamol sulphate and beclomethasone dipropionate. Other agents that can be employed include carbohydrates, such as heparin.

The active agent can be for systemic absorption and administerable via the lungs, buccal mucosa and/or nasal cavity. Examples include peptides and polypeptides, such as Dnase, leukotrienes and insulin, analgesics such as fentanyl and dihydroergotamine, anti-cancer agents, anti-viral agents and antibiotics. Further such active agents include apomorphine.

The present invention can be carried out with any pharmaceutically active agent. The preferred active agents include:

v) steroid drugs such as alcometasone, beclomethasone, beclomethasone dipropionate, betamethasone, budesonide, ciclesonide, clobetasol, deflazacort, diflucortolone, desoxymethasone, dexamethasone, fludrocortisone, flunisolide, fluocinolone, fluometholone, fluticasone, fluticasone proprionate, hydrocortisone, triamcinolone, nandrolone decanoate, neomycin sulphate, rimexolone, methylprednisolone and prednisolone;

2) bronchodilators such as β₂-agonists including salbutamol, formoterol, salmeterol, fenoterol, bambuterol, bitolterol, sibenadet, metaproterenol, epinephrine, isoproterenol, pirbuterol, procaterol, terbutaline and isoetharine antimuscarinics including ipratropium and tiotropium, and xanthines including aminophylline and theophylline;

3) nitrates such as isosorbide mononitrate, isosorbide dinitrate and glyceryl trinitrate;

4) antihistamines such as azelastine, chlorpheniramine, astemizole, cetirizine, cinnarizine, desloratadine, loratadine, hydroxyzine, diphenhydramine, fexofenadine, ketotifen, promethazine, trimeprazine and terfenadine;

5) anti-inflammatory agents such as piroxicam, nedocromil, benzydamine, diclofenac sodium, ketoprofen, ibuprofen, heparinoid, cromoglycate, fasafungine, iodoxamide and p38 MAP kinase inhibitors;

6) anticholinergic agents such as atropine, benzatropine, biperiden, cyclopentolate, oxybutinin, orphenadine, glycopyrronium, glycopyrrolate, procyclidine, propantheline, propiverine, tiotropium, trihexyphenidyl, tropicamide, trospium, ipratropium bromide and oxitroprium bromide;

7) leukotriene receptor antagonists such as montelukast and zafirlukast;

8) anti-allergies such as ketotifen;

9) anti-emetics such as bestahistine, dolasetron, nabilone, prochlorperazine, ondansetron, trifluoperazine, tropisetron, domperidone, hyoscine, cinnarizine, metoclopramide, cyclizine, dimenhydrinate and promethazine;

10) hormonal drugs (including hormone analogues) such as lanreotide, octreotide, insulin, pegvisomant, protirelin, thyroxine, salcotonin, somatropin, tetracosactide, vasopressin and desmopressin;

11) sympathomimetic drugs such as adrenaline, noradrenaline, dexamfetamine, dipirefin, dobutamine, dopexamine, phenylephrine, isoprenaline, dopamine, pseudoephedrine, tramazoline and xylometazoline;

12) opioids, preferably for pain management, such as buprenorphine, dextromoramide, dextropropoxypene, diamorphine, codeine, dextropropoxyphene, dihydrocodeine, hydromorphone, papaveretum, pholcodeine, loperamide, fentanyl, methadone, morphine, oxycodone, phenazocine, pethidine, tramadol and combinations thereof with an anti-emetic;

13) analgesics such as aspirin and other salicylates, paracetamol, clonidine, codine, coproxamol, ergotamine, gabapentin, pregabalin, sumatriptan, and non-steroidal anti-inflammatory drugs (NSAIDs) including celecoxib, etodolac, etoricoxib and meloxicam;

14) acetylcholinesterase inhibitors such as donepezil, galantamine and rivastigmine;

15) immunomodulators such as interferon (e.g. interferon beta-Ia and interferon beta-Ib) and glatiramer;

16) NMDA receptor antagonists such as mementine;

17) hypoglycaemics such as sulphonylureas including glibenclamide, gliclazide, glimepiride, glipizide and gliquidone, biguanides including metformin, thiazolidinediones including pioglitazone, rosiglitazone, nateglinide, repaglinide and acarbose;

18) narcotic agonists and opiate antidotes such as naloxone, and pentazocine;

19) phosphodiesterase inhibitors such as non-specific phosphodiesterase inhibitors including theophylline, theobromine, IBMX, pentoxifylline and papaverine; phosphodiesterase type 3 inhibitors including bipyridines such as milrinone, amrinone and olprinone; imidazolones such as piroximone and enoximone; imidazolines such as imazodan and 5-methyl-imazodan; imidazo-quinoxalines; and dihydropyridazinones such as indolidan and LY181512 (5-(6-oxo-1,4,5,6-tetrahydro-pyridazin-3-yl)-1,3-dihydro-indol-2-one); dihydroquinolinone compounds such as cilostamide, cilostazol, and vesnarinone; phosphodiesterase type 4 inhibitors such as cilomilast, etazolate, rolipram, roflumilast and zardaverine, and including quinazolinediones such as nitraquazone and nitraquazone analogs; xanthine derivatives such as denbufylline and arofylline; tetrahydropyrimidones such as atizoram; and oxime carbamates such as filaminast; and phosphodiesterase type 5 inhibitors including sildenafil, zaprinast, vardenafil, tadalafil, dipyridamole, and the compounds described in WO 01/19802, particularly (S)-2-(2-hydroxymethyl-1-pyrrolidinyl)-4-(3-chloro-4-methoxy-benzylamino)-5-[N-(2-pyrimidinylmethyl)carbamoyl]pyrimidine, 2-(5,6,7,8-tetrahydro-1,7-naphthyridin-7-yl)-4-(3-chloro-4-methoxybenzylamino)-5-[N-(2-morpholinoethyl)carbamoyl]-pyrimidine, and (S)-2-(2-hydroxymethyl-1-pyrrolidinyl)-4-(3-chloro-4-methoxy-benzylamino)-5-[N-(1,3,5-trimethyl-4-pyrazolyl)carbamoyl]-pyrimidine);

20) antidepressants such as tricyclic and tetracyclic antidepressants including amineptine, amitriptyline, amoxapine, butriptyline, cianopramine, clomipramine, dosulepin, doxepin, trimipramine, clomipramine, lofepramine, nortriptyline, tricyclic and tetracyclic amitryptiline, amoxapine, butriptyline, clomipramine, demexiptiline, desipramine, dibenzepin, dimetacrine, dothiepin, doxepin, imipramine, iprindole, levoprotiline, lofepramine, maprotiline, melitracen, metapramine, mianserin, mirtazapine, nortryptiline, opipramol, propizepine, protriptyline, quinupramine, setiptiline, tianeptine and trimipramine; selective serotonin and noradrenaline reuptake inhibitors (SNRIs) including clovoxamine, duloxetine, milnacipran and venlafaxine; selective serotonin reuptake inhibitors (SSRIs) including citalopram, escitalopram, femoxetine, fluoxetine, fluvoxamine, ifoxetine, milnacipran, nomifensine, oxaprotiline, paroxetine, sertraline, sibutramine, venlafaxine, viqualine and zimeldine; selective noradrenaline reuptake inhibitors (NARIs) including demexiptiline, desipramine, oxaprotiline and reboxetine; noradrenaline and selective serotonin reuptake inhibitors (NASSAs) including mirtazapine; monoamine oxidase inhibitors (MAOIs) including amiflamine, brofaromine, clorgyline, α-ethyltryptamine, etoperidone, iproclozide, iproniazid, isocarboxazid, mebanazine, medifoxamine, moclobemide, nialamide, pargyline, phenelzine, pheniprazine, pirlindole, procarbazine, rasagiline, safrazine, selegiline, toloxatone and tranylcypromine; muscarinic antagonists including benactyzine and dibenzepin; azaspirones including buspirone, gepirone, ipsapirone, tandospirone and tiaspirone; and other antidepressants including amesergide, amineptine, benactyzine, bupropion, carbamazepine, fezolamine, flupentixol, levoprotiline, maprotiline, medifoxamine, methylphenidate, minaprine, nefazodone, nomifensine, oxaflozane, oxitriptan, rolipram, sibutramine, teniloxazine, tianeptine, tofenacin, trazadone, tryptophan, viloxazine, and lithium salts;

21) serotonin agonists such as 2-methyl serotonin, buspirone, ipsaperone, tiaspirone, gepirone, lysergic acid diethylamide, ergot alkaloids, 8-hydroxy-(2-N,N-dipropylamino)-tetraline, 1-(4-bromo-2,5-dimethoxyphenyl)-2-aminopropane, cisapride, sumatriptan, m-chlorophenylpiperazine, trazodone, zacopride and mezacopride;

22) serotonin antagonists including ondansetron, granisetron, metoclopramide, tropisetron, dolasetron, trimethobenzamide, methysergide, risperidone, ketanserin, ritanserin, clozapine, amitryptiline, R(+)-α-(2,3-dimethoxyphenyl)-1-[2-(4-fluorophenyl)ethyl]-4-piperidine-methanol, azatadine, cyproheptadine, fenclonine, dexfenfluramine, fenfluramine, chlorpromazine and mianserin;

23) adrenergic agonists including methoxamine, methpentermine, metaraminol, mitodrine, clonidine, apraclonidine, guanfacine, guanabenz, methyldopa, amphetamine, methamphetamine, epinephrine, norepinephrine, ethylnorepinephrine, phenylephrine, ephedrine, pseudo-ephedrine, methylphenidate, pemoline, naphazoline, tetrahydrozoline, oxymetazoline, xylometazoline, phenylpropanolamine, phenylethylamine, dopamine, dobutamine, colterol, isoproterenol, isotharine, metaproterenol, terbutaline, metaraminol, tyramine, hydroxyamphetamine, ritodrine, prenalterol, albuterol, isoetharine, pirbuterol, bitolterol, fenoterol, formoterol, procaterol, salmeterol, mephenterine and propylhexedrine;

24) adrenergic antagonists such as phenoxybenzamine, phentolamine, tolazoline, prazosin, terazosin, doxazosin, trimazosin, yohimbine, ergot alkaloids, labetalol, ketanserin, urapidil, alfuzosin, bunazosin, tamsulosin, chlorpromazine, haloperidol, phenothiazines, butyrophenones, propranolol, nadolol, timolol, pindolol, metoprolol, atenolol, esmolol, acebutolol, bopindolol, carteolol, oxprenolol, penbutolol, carvedilol, medroxalol, naftopidil, bucindolol, levobunolol, metipranolol, bisoprolol, nebivolol, betaxolol, carteolol, celiprolol, sotalol, propafenone and indoramin;

25) adrenergic neurone blockers such as bethanidine, debrisoquine, guabenxan, guanadrel, guanazodine, guanethidine, guanoclor and guanoxan;

26) benzodiazepines such as alprazolam, bromazepam, brotizolam, chlordiazepoxide, clobazam, clonazepam, clorazepate, demoxepam, diazepam, estazolam, flunitrazepam, flurazepam, halazepam, ketazolam, loprazolam, lorazepam, lormetazepam, medazepam, midazolam, nitrazepam, nordazepam, oxazepam, prazepam, quazepam, temazepam and triazolam;

27) mucolytic agents such as N-acetylcysteine, recombinant human DNase, amiloride, dextrans, heparin, desulphated heparin and low molecular weight heparin;

28) antibiotic and antibacterial agents such as metronidazole, sulphadiazine, triclosan, neomycin, amoxicillin, amphotericin, clindamycin, aclarubicin, dactinomycin, nystatin, mupirocin and chlorhexidine;

29) anti-fungal drugs such as caspofungin, voriconazole, polyene antibiotics including amphotericin, and nystatin, imidazoles and triazoles including clotrimazole, econazole nitrate, fluconazole, ketoconazole, itraconazole, terbinafine and miconazole;

30) antivirals such as oseltamivir, zanamivir, amantadine, inosine pranobex and palivizumab, DNA polymerase inhibitors including aciclovir, adefovir and valaciclovir, nucleoside analogues including famiciclovir, penciclovir and idoxuridine and interferons;

31) vaccines;

32) immunoglobulins;

33) local anaesthetics such as amethocaine, bupivacaine, hydrocortisone, methylprednisolone, prilocaine, proxymetacaine, ropivacaine, tyrothricin, benzocaine and lignocaine;

34) anticonvulsants such as sodium valproate, carbamazepine, oxcarbazepine, phenytoin, fosphenytoin, diazepam, lorazepam, clonazepam, clobazam, primidone, lamotrigine, levetiracetam, topiramate, gabapentin, pregabalin, vigabatrin, tiagabine, acetazolamide, ethosuximide and piracetam;

35) angiotensin converting enzyme inhibitors such as captopril, cilazapril, enalapril, fosinopril, imidapril hydrochloride, lisinopril, moexipril hydrochloride, perindopril, quinapril, ramipril and trandolapril;

36) angiotension II receptor blockers, such as candesartan, cilexetil, eprosartan, irbesartan, losartan, olmesartan medoxomil, telmisartan and valsartan;

37) calcium channel blockers such as amlodipine, bepridil, diltiazem, felodipine, flunarizine, isradipine, lacidipine, lercanidipine, nicardipine, nifedipine, nimodipine and verapamil;

38) alpha-blockers such as indoramin, doxazosin, prazosin, terazosin and moxisylate;

39) antiarrhythmics such as adenosine, propafenone, amidodarone, flecainide acetate, quinidine, lidocaine hydrochloride, mexiletine, procainamide and disopyramide;

40) anti-clotting agents such as aspirin, heparin and low molecular weight heparin, epoprostenol, dipyridamole, clopidogrel, alteplase, reteplase, streptokinase, tenecteplase, certoparin, heparin calcium, enoxaparin, dalteparin, danaparoid, fondaparin, lepirudin, bivalirudin, abciximab, eptifibatide, tirofiban, tinzaparin, warfarin, lepirudin, phenindione and acenocoumarol;

41) potassium channel modulators such as nicorandil, cromakalim, diazoxide, glibenclamide, levcromakalim, minoxidil and pinacidil;

42) cholesterol-lowering drugs such as colestipol, colestyramine, bezafibrate, fenofibrate, gemfibrozil, ciprofibrate, rosuvastatin, simvastatin, fluvastatin, atorvastatin, pravastatin, ezetimibe, ispaghula, nictotinic acid, acipimox and omega-3 triglycerides;

43) diuretics such as bumetanide, furosemide, torasemide, spironolactone, amiloride, bendroflumethiazide, chlortalidone, metolazone, indapamide and cyclopenthiazide;

44) smoking cessation drugs such as nicotine and bupropion;

45) bisphosphonates such as alendronate sodium, sodium clodronate, etidronate disodium, ibandronic acid, pamidronate disodium, isedronate sodium, tiludronic acid and zoledronic acid;

46) dopamine agonists such as amantadine, bromocriptine, pergolide, cabergoline, lisuride, ropinerole, pramipexole and apomorphine;

47) nucleic-acid medicines such as oligonucleotides, decoy nucleotides, antisense nucleotides and other gene-based medicine molecules;

48) antipsychotics such as: dopamine antagonists including chlorpromazine, prochlorperazine, fluphenazine, trifluoperazine and thioridazine; phenothiazines including aliphatic compounds, piperidines and piperazines; thioxanthenes, butyrophenones and substituted benzamides; atypical antipsychotics including clozapine, risperidone, olanzapine, quetiapine, ziprasidone, zotepine, amisulpride and aripiprazole; and

49) pharmaceutically acceptable salts or derivatives of any of the foregoing.

In preferred embodiments of the present invention, the active agent is heparin (fractionated and unfractionated), apomorphine, clobazam, clomipramine or glycopyrrolate.

In addition, the active agents used in the present invention may be small molecules, proteins, carbohydrates or mixtures thereof.

The particles of active agent can include, consist or consist essentially of a plurality of pharmaceutically active compounds, so long as the compounds are chemically compatible.

Formulations in accordance with the present invention can include mixtures of first composite particles that include a first pharmaceutically active agent and second composite particles that include a second pharmaceutically active agent. In a preferred embodiment of the invention, wherein the formulation includes a plurality of pharmaceutically active agents, at least one such agent is a β₂-agonist and another is a steroid. Other suitable mixtures include those comprising two different β₂-agonists, such as salbutamol in combination with salmeterol, or two different steroids.

The propellant preferably comprises, or consists or consists essentially of an HFA, or mixture of HFAs. The preferred HFAs are HFA-134a and HFA-227, with the former being most preferred. The formulation is preferably substantially free of CFC. The formulation can include additional gases, such as carbon dioxide or an oxide of nitrogen.

The formulation can include a co-solvent, such as ethanol, but it is preferred for the formulation to include less than 3, 2, 1, 0.5, 0.1, 0.01 or 0.001% of any co-solvent, particularly a polar co-solvent and especially ethanol. In particular, it is preferred for the formulation to be substantially or essentially free of any such co-solvent, especially ethanol.

In further preferred embodiments, formulations in accordance with the invention include less than 3, 2, 1, 0.5, 0.1, 0.01 or 0.001%, or are substantially or essentially free of dissolved dispersing agent and, in particular, are substantially or essentially free of PVP (poly vinyl pyrolidone).

In yet further preferred embodiments, formulations in accordance with the invention consist or consist essentially of liquefied propellant gas and composite particles that comprise, or consist or consist essentially of, pharmaceutically active agent and dispersing agent.

Preferably, the coating or shell of dispersing agent formed around the particles of active agent has a mean thickness of 1, 0.5 or 0.2 μm or less.

It is further preferred that formulations in accordance with the invention cannot consist of HFA 134a and composite particles that consist of apomorphine and lecithin. It is also preferred for formulations in accordance with the invention not to comprise composite particles that comprise apomorphine and lecithin.

The particles of fused active agent and dispersing agent preferably have an MMAD of between 0.1 and 10 μm. In embodiments where the formulation is for delivery to the deep lung, the particles of fused active agent and dispersing agent have an MMAD of up to about 10 μm. In embodiments where the formulation is for delivery to locations other than the deep lung, the particles of fused active agent and dispersing agent have an MMAD more than about 10 μm.

In preferred embodiments where the formulation is for delivery to the lung, the MMAD of the composite particles of fused active agent and dispersing agent is no more than 5, 3 or 1 μm. Advantageously, at least 90% by weight of the composite particles have a diameter of not more than 10, 5, 3, 2.5, 2, 1.5 or 1 μm. In detail, the particles should have an MMAD in the range of 3 to 0.1 or 0.05 μm for absorption in the deep lung, 5 to 2 or 0.5 μm for absorption in the respiratory bronchioles, 10 to 2 μm for delivery to the higher respiratory system and 2 to 0.05 μm for delivery to the alveoli. Accordingly, advantageously at least 90% by weight of the composite particles can have an aerodynamic diameter in the range of 3 to 0.1 or 0.05 m, preferably 5 to 2 or 0.5 μm, advantageously 10 to 2 μm and especially advantageously 2 to 0.05 μm. The MMAD of the composite particles will not normally be lower than 0.01 μm.

It can be desirable to have a formulation wherein the size distribution of the composite particles is as narrow as possible. For example, the geometric standard deviation of the composite particle aerodynamic or volumetric size distribution (σg) is preferably not more than 2, more preferably not more than 1.8, not more than 1.6, not more than 1.5, not more than 1.4, or even not more than 1.2. This will improve dose efficiency and reproducibility.

In the practice of methods in accordance with the invention the mechanical energy required in order to fuse the dispersing agents to the surface of particles of pharmaceutically active agent can be applied by a milling process carried out in a suitable milling device. The milling conditions, for example, the intensity of milling and duration, should be selected to provide the required degree of energy. Ball milling is one preferred method; centrifugal and planetary ball milling being preferred examples. Milling can be performed in a high energy media mill or an agitator bead mill, for example, the Netzch high energy media mill, or the DYNO-mill (Willy A. Bachofen A G, Switzerland). However the most preferred milling techniques include those described in R. Pfeffer et al. “Synthesis of engineered particulates with tailored properties using dry particle coating”, Powder Technology 117 (2001) 40-67. These include processes using the MechanoFusion® machine, the Hybidizer® machine, the Theta Composer®, magnetically assisted impaction processes and rotating fluidized bed coaters. Cyclomix methods may also be used.

Preferably, the technique employed to apply the required mechanical energy involves the compression of a mixture of particles of the dispersing agent and particles of the pharmaceutically active agent in a nip formed between two portions of a milling machine, as is the case in the MechanoFusion® and Cyclomix devices.

Some preferred milling methods will now be described in greater detail:

### MechanoFusion®:

As the name suggests, this dry coating process is designed to mechanically fuse a first material onto a second material. The first material is generally smaller and/or softer than the second. The MechanoFusion and Cyclomix working principles are distinct from alternative milling techniques in having a particular interaction between inner element and vessel wall, and are based on providing energy by a controlled and substantial compressive force.

The fine active particles and the particles of dispersing agent are fed into the MechanoFusion driven vessel, where they are subject to a centrifugal force and are pressed against the vessel inner wall. The powder is compressed between the fixed clearance of the drum wall and a curved inner element with high relative speed between drum and element. The inner wall and the curved element together form a gap or nip in which the particles are pressed together. As a result the particles experience very high shear forces and very strong compressive stresses as they are trapped between the inner drum wall and the inner element (which has a greater curvature than the inner drum wall). The particles violently collide against each other with enough energy to locally heat and soften, break, distort, flatten and wrap the particles of dispersing agent around the core particle to form a coating. The energy is generally sufficient to break up agglomerates and some degree of size reduction of both components may occur. Embedding and fusion of additive particles of dispersing agent onto the active particles may occur, and may be facilitated by the relative differences in hardness (and optionally size) of the two components. Either the outer vessel or the inner element may rotate to provide the relative movement. The gap between these surfaces is relatively small, and is typically less than 10 mm and is preferably less than 5 mm, more preferably less than 3 mm. This gap is fixed, and consequently leads to a better control of the compressive energy than is provided in some other forms of mill such as ball and media mills. Also, in general, no impaction of milling media surfaces is present so that wear and consequently contamination are minimised. The speed of rotation may be in the range of 200 to 10,000 rpm. A scraper may also be present to break up any caked material building up on the vessel surface. This is particularly advantageous when using fine cohesive starting materials. The local temperature may be controlled by use of a heating/cooling hacked built into the drum vessel walls. The powder may be re-circulated through the vessel.

### Cyclomix Method (Hosokawa Microm):

The cyclomix comprises a stationary conical vessel with a fast rotating shaft with paddles which move close to the wall. Due to the high rotational speed of the paddles, the powder is propelled towards the wall, and as a result the mixture experiences very high shear forces and compressive stresses between wall and paddle. Such effects are similar to those in MechanoFusion as described above and may be sufficient to locally heat and soften, to break, distort, flatten and wrap the particles of dispersing agent around the active particles to form a coating. The energy is sufficient to break up agglomerates and some degree of size reduction of both components may also occur depending on the conditions and upon the size and nature of the particles.

### Hybridiser® Method:

This is a dry process which can be described as a product embedding or filming of one powder onto another. The fine active particles and fine or ultra fine particles of dispersing agent are fed into a conventional high shear mixer pre-mix system to form an ordered mixture. This powder is then fed into the Hybridiser. The powder is subjected to ultra-high speed impact, compression and shear as it is impacted by blades on a high speed rotor inside a stator vessel, and is re-circulated within the vessel. The active and additive particles collide with each other. Typical speeds of rotation are in the range of 5,000 to 20,000 rpm. The relatively soft fine particles of dispersing agent experience sufficient impact force to soften, break, distort, flatten and wrap around the active particle to form a coating. There may also be some degree of embedding into the surface of the active particles.

As noted, other preferred methods include ball and high energy media mills which are also capable of providing the desired high shear force and compressive stresses between surfaces. However, as the clearance gap is not controlled, the coating process may be less well controlled than it is in MechanoFusion and some problems such as a degree of undesired re-agglomeration may occur. These media mills may be rotational, vibrational, agitational, centrifugal or planetary in nature.

In a third aspect, the present invention provides a medical device for delivering a pharmaceutical formulation in aerosol or spray form, comprising a pharmaceutical formulation in accordance with the first aspect of the invention. Preferably the device is a conventional pMDI, for example a device such as those disclosed in WO92/11190, U.S. Pat. No. 4,819,834 and U.S. Pat. No. 4,407,481. The device can be adapted for pulmonary, nasal, buccal or dermal delivery of the formulation. Preferably, the device is adapted for pulmonary delivery of the formulation.

Wherever an entity is herein described as including or comprising a particular ingredient or component, or plurality of ingredients or components, it can, in embodiments, consist or consist essentially of the identified ingredient(s) or component(s).

### EXAMPLE 1

### Anderson Cascade Impactor Study

The impactor study was carried out using 50 μl valves on CCL coated cans. Each can was made up to give a 100 μg dose in 50 μl. The only components in the final pMDIs were the MechanoFused formulation and HFA 134a propellant. See below for details of the preparation of the formulations and filled cans.

Five shots were fired to waste prior to testing and ten shots were performed per test. A single test only was carried out for each formulation. Flow rate 28.31/min. The results are summarized below:

| | | | | | Fine | Fine | |
|---|---|---|---|---|---|---|---|
| | | | Metered | Delivered | Particle | Particle | |
| | | Actuator | Dose | Dose | Dose* | Fraction* | MMAD |
| Formulation | Composition | (mm) | (μg) | (μg) | (μg) | (%) | (μm) |
| VPR030818HHA | 25% S PC-3 | 0.30 | 113.6 | 87.9 | 40.4 | 46.0 | 4.3 |
| VPR030819HHA | 10% S 100-3 | 0.42 | 113.7 | 96.3 | 34.5 | 35.8 | 4.1 |
| VPR030819HHB | 25% S 100-3 | 0.30 | 116.8 | 96.7 | 38.9 | 40.2 | 3.9 |

*Fine particle dose and fraction refer to amount less than 5 μm.

### Twenty Shot Through Life Study

Shot weights were tested for VPR030818HHA in can UFP030902SBA using a 0.30 mm actuator and a 0.42 mm actuator to address concerns that the actuator orifice might be prone to blocking.

0.30 mm actuator:

Shots 21 to 40, mean shot weight 0.0637 g, standard deviation 0.001437, RSD 2.25%.

0.42 mm actuator:

Shots 46 to 65, mean shot weight 0.0646 g, standard deviation 0.000976, RSD 1.51%.

### Density Measurements

Approximately 5 g of material was placed in a measuring cylinder and the volume noted. The poured density was calculated. The measuring cylinder was tapped 100 times and the volume noted. Tapping was repeated to constant volume and the tapped density calculated. The equipment used was a Stampf Volumeter STA V2003.

| | | | Number |
|---|---|---|---|
| | Poured | Tapped Density | of Taps to |
| Formulation | Density (g/ml) | (g/ml) | Constant Volume |
| Salbutamol Sulphate | 0.19 | 0.24 | 200 |
| VPR030818HHA | 0.25 | 0.38 | 400 |
| VPR030819HHA | 0.25 | 0.42 | 400 |
| VPR030819HHB | 0.28 | 0.42 | 300 |

### Preparations of Powder Formulations

### Materials Used:

Salbutamol sulphate (Micron Technologies batch 019744)

Lecithin S PC-3 (Lipoid batch 256113- 1/14 prepared in Grindomix at 3000 rpm for 1 minute)

Lecithin S 100-3 (Lipoid batch 2540565-1 prepared in Grindomix at 3000 rpm for 1 minute)

### Equipment Used:

Hosokawa AMS-MINI with 1 mm gap rotor (MechanoFusion®))

Retsch Grindomix GM200

### VPR030818HHA

### Powderpreparation:

12.0 g salbutamol sulphate and 4.0 g lecithin S PC-3 were weighed into a beaker. The powder was transferred to the Hosokawa AMS-MINI via a funnel attached to the largest port in the lid with the equipment running at 3.5%. The port was sealed and the cooling water switched on. The equipment was run at 20% for 5 minutes followed by 50% for 10 minutes. The equipment was switched off, dismantled and the resulting formulation recovered mechanically. Recovery was 91% by weight.

### VPR030819HHA

### Powderpreparation:

14.4 g salbutamol sulphate and 1.6 g lecithin S 100-3 were weighed into a beaker. The powder was transferred to the Hosokawa AMS-MINI via a funnel attached to the largest port in the lid with the equipment running at 3.5%. The port was sealed and the cooling water switched on. The equipment was run at 20% for 5 minutes followed by 50% for 10 minutes. The equipment was switched off, dismantled and the resulting formulation recovered mechanically. Recovery was 88% by weight.

### VPR030819HHB

### Powderpreparation:

14.0 g salbutamol sulphate and 4.0 g lecithin S 100-3 were weighed into a beaker. The powder was transferred to the Hosokawa AMS-MINI via a funnel attached to the largest port in the lid with the equipment running at 3.5%. The port was sealed and the cooling water switched on. The equipment was run at 20% for 5 minutes followed by 50% for 10 minutes. The equipment was switched off, dismantled and the resulting formulation recovered mechanically. Recovery was 92% by weight.

### Preparation of Cans

### VPR030818HHA

0.0265 g powder was weighed into the can,

a 50 μl valve was crimped to the can,

12.2710 g HFA 134a was weighed into the can.

### VPR030819HHA

0.0222 g powder was weighed into the can,

a 50 μl valve was crimped to the can,

12.2040 g HFA 134a was weighed into the can.

### VPR030819HHB

0.0268 g powder was weighed into the can,

a 50 μl valve was crimped to the can,

12.2121 g HFA 134a was weighed into the can.

### EXAMPLE 2

Stability tests on MechanoFused Salbutamol pMDI Formulations for stability
Production of suspension pMDI formulations:

### VPR030818HHA

### Suspension:

0.0267 g of powder were weighed into a glass canister, a continuous flow Bespak valve was crimped onto the canister and 12.2 g HFA 134a were injected under pressure. The canister was placed in an ultrasonic bath and sonicated for 10 minutes.

### VPR030819HHA

### Suspension:

0.0222 g of powder were weighed into a glass canister, a continuous flow Bespak valve was crimped onto the canister and 12.2 g HFA 134a were injected under pressure. The canister was placed in an ultrasonic bath and sonicated for 10 minutes.

### VPR030819HHB

### Suspension:

0.0267 g of powder were weighed into a glass canister, a continuous flow Bespak valve was crimped onto the canister and 12.2 g HFA 134a were injected under pressure. The canister was placed in an ultrasonic bath and sonicated for 10 minutes.

### Methodology

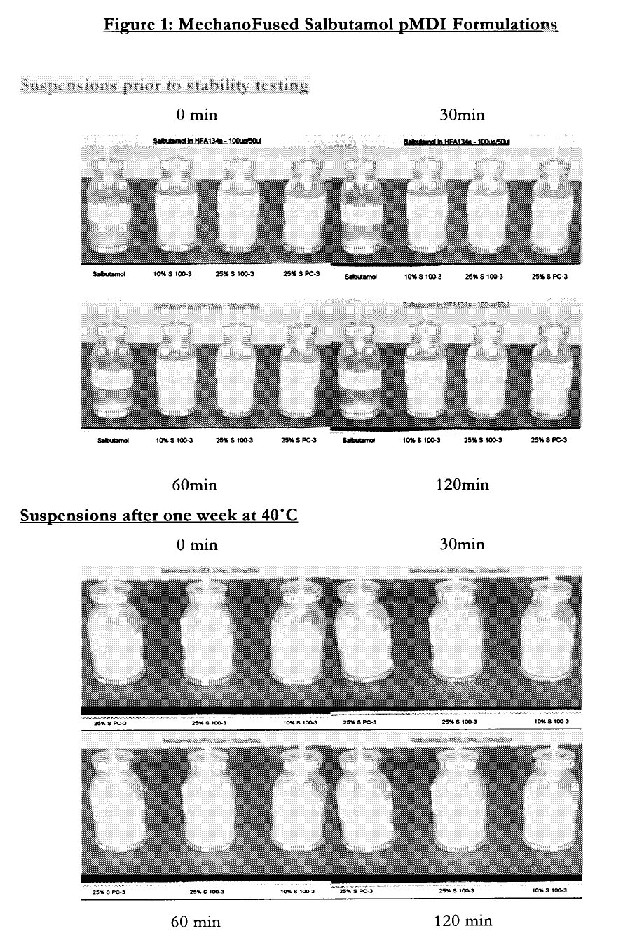

The glass canisters were lightly shaken and photographed after 0, 30, 60, 120 minutes. They were then stored at 40° C. for 1 week, lightly shaken and photographed after 0, 30, 60, 120 minutes. The photographs are set out in FIG. 1.

As can be seen in FIG. 1, no significant changes in the formulations were observed after storage at 40° C. for 1 week.

### EXAMPLE 3

### Dispersion Testing Using Malvern Mastersizer

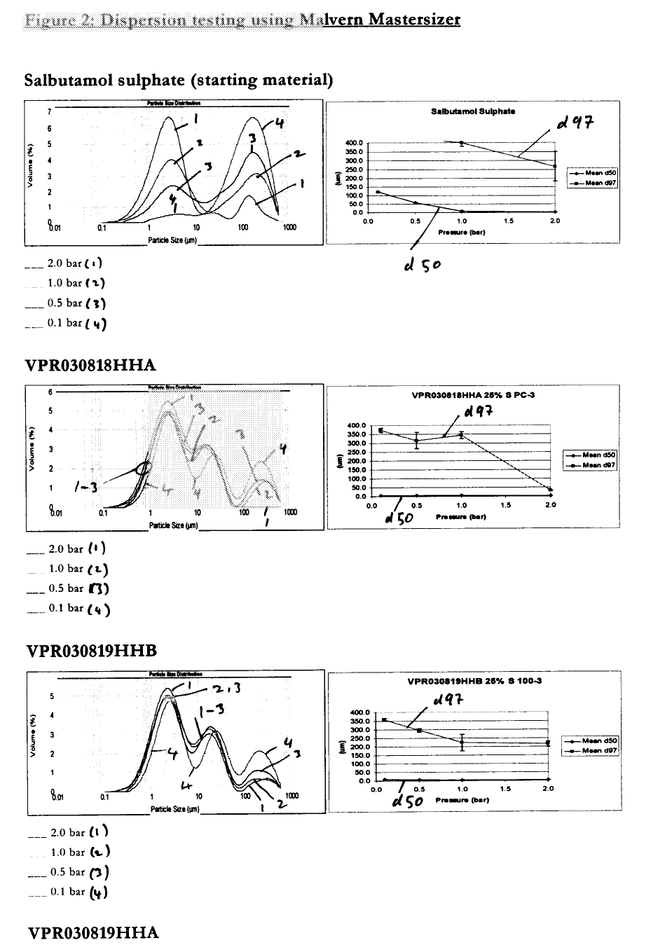

Each powder was separately dispersed at 2, 1, 0.5 and 0.1 bar in the Malvern Scirocco disperser and analysed using a Malvern Mastersizer 2000. A representative dispersion plot for each pressure was overlaid to give one dispersion graph per formulation. The d50 and d97 values were also plotted versus the dispersing pressure. See FIG. 2.

AU the formulations showed better dispersion at all pressures with lower values for d50 and d97 at all pressures when compared with the starting material (Salbutamol sulphate).

### EXAMPLE 4

### Materials

Salbutamol sulphate was obtained in a micronised form. The dispersing agents (DAs) were as follows: L-leucine was supplied from Ajimoto Co., lecithin (SPC-3) from Lipoid GmbH and magnesium stearate from Avocado. All were used as supplied.

### Preparation of Powder Formulations

Blends of the drug and DAs (5% w/w FCA) were prepared using a Mechanofusion system using the Mini Kit with a rotor gap of 1 mm (Hosokawa-Alpine, Augsburg, Germany). Powders to be processed were sealed into the Mechanofusion system core. A cold-water circulation assured the regulation of the internal vessel temperature using an incorporated water jacket. Samples were blended for 10 minutes at 80% full speed (˜5000 rpm) to mechanically fuse the DA to the micronised drug.

Preparation of pMDIs

The powders comprising pure micronised salbutamol sulphate drug or drug mechanofused with leucine, lecithin or magnesium stearate were measured into pMDI cans. Metering valves were clamped onto the cans, and these were back filled with HFA 134a propellant. Each can was shaken vigorously to generate a dispersion.

In Vitro Measurement of pMDIs

An Andersen cascade impactor was used to characterise the aerosol plumes generated from each of the 4 different suspension pMDIs. Air-flow of 28.3 litres per minute was drawn through the impactor, and 10 repeated shots were fired. Each pMDI was shaken and weighed in between each actuation. The drug deposited on each stage of the impactor, as well as drug on the device, throat and rubber mouthpiece adaptor was collected into a solvent, and quantified by HPLC. The determination was repeated 3 times for each of the 4 suspensions.

Values for metered dose (MD), emitted dose (ED), fine particle dose (FPD) and fine particle fraction of emitted dose (FPF) were measured. FPD was taken as the cumulative dose collected on stage 3 and below, and FPF was the FPD divided by ED expressed as a percentage.

### Resuits:

| | Composition | | | |
|---|---|---|---|---|
| | MD (μg) | ED (μg) | FPD (μg) | FPF(%) |
| Drug only | 98 | 88 | 0 | 0 |
| With leucine | 240 | 209 | 74 | 35 |
| With lecithin | 356 | 311 | 116 | 37 |
| With magnesium stearate | 238 | 206 | 111 | 53 |

### EXAMPLE 5

This example describes the preparation of various composite particle powders suitable for use in formulations in accordance with the invention. These powders can be used to produce pMDIs in the manner described in any of examples 1,2 and 4.

### EXAMPLE 5.1

### Mechanofused Budesonide with Magnesium Stearate

Blends of magnesium stearate and budesonide were prepared by Mechanofusion using the Hosokawa AMS-MINI, with blending being carried out for 60 minutes at approximately 4000 rpm. The magnesium stearate used was a standard grade supplied by Avocado Research Chemicals Ltd. The budesonide was micronised.

Blends of budesonide and magnesium stearate were prepared at different weight percentages of magnesium stearate. Blends of 5% w/w and 10% w/w, were prepared.

### EXAMPLE 5.2

### Preparation of Mechanofused Clomipramine with Magnesium Stearate

20 g of a mix comprising micronised clomipramine and 2% magnesium stearate was weighed into the Hosokawa AMS-MINI Mechanofusion system via a funnel attached to the largest port in the lid with the equipment running at 3.5%. The port was sealed and the cooling water switched on. The equipment was run at 20% for 5 minutes followed by 80% for 10 minutes. The equipment was switched off, dismantled and the resulting formulation recovered mechanically.

### EXAMPLE 5.3

### Mechanofused Apomorphine and Leucine

15 g of micronised apomorphine and 0.75 g leucine are weighed into the Hosokawa AMS-MINI Mechanofusion system via a funnel attached to the largest port in the lid with the equipment running at 3.5%. The port is sealed and the cooling water switched on. The equipment is run at 20% for 5 minutes followed by 80% for 10 minutes. The equipment is then switched off, dismantled and the resulting formulation recovered mechanically.

### EXAMPLE 5.4

### Mechanofused Clomipramine and Magnesium Stearate

20 g of a mix comprising 95% micronised clomipramine and 5% magnesium stearate are weighed into the Hosokawa AMS-MINI Mechanofusion system via a funnel attached to the largest port in the lid with the equipment running at 3.5%. The port is sealed and the cooling water switched on. The equipment is run at 20% for 5 minutes followed by 80% for 10 minutes. The equipment is then switched off, dismantled and the resulting formulation recovered mechanically.

### EXAMPLE 5.5

### Mechanofused Theophylline and Magnesium Stearate

20 g of a mix comprising 95% micronised theophylline and 5% magnesium stearate are weighed into the Hosokawa AMS-MINI Mechanofusion system via a funnel attached to the largest port in the lid with the equipment running at 3.5%. The port is sealed and the cooling water switched on. The equipment is run at 20% for 5 minutes followed by 80% for 10 minutes. The equipment is then switched off, dismantled and the resulting formulation recovered mechanically.

The active agent used in this example, theophylline, may be replaced by other phosphodiesterase inhibitors, including phosphodiesterase type 3, 4 or 5 inhibitors, as well as other non-specific ones.

### EXAMPLE 5.6

### Jet Milled Clomipramine and Magnesium Stearate

20 g of a mix comprising 95% micronised clomipramine and 5% magnesium stearate are co-jet milled in a Hosokawa AS50 jet mill.

A number of micronised drugs are co-jet milled with magnesium stearate for the purposes of replacing the clomipramine in this example. These micronised drugs included budesonide, formoterol, salbutamol, glycopyrrolate, heparin, insulin and clobazam. Further compounds are considered suitable, including the classes of active agents and the specific examples listed above.

### EXAMPLE 5.7

### Jet Milled Bronchodilator and Magnesium Stearate

20 g of a mix comprising 95% micronised bronchodilator drug and 5% magnesium stearate are co-jet milled in a Hosokawa AS50 jet mill.

### EXAMPLE 6

### Surface Chemical Analysis of Powders

The aim of the analysis is to identify the presence of magnesium stearate on the surface of a model of a co-micronised active agent. The model powders were processed in two different ways, with one representing a conventional pharmaceutical blending process, and the other being the intensive Mechanofusion process which is the subject of the invention. The aim was to show the contrast in surface coating efficiency. The model material, representing the micronised active agent particles, was micronised lactose.

The powders have been analyzed using both TOF-SIMS and XPS. TOF-SIMS provides a mass spectrum of the outermost 1 nm of the surface, and is used here to asses whether the magnesium stearate coverage of the lactose is complete or in patches. XPS provides a spectrum representative of the outermost 10 nm of the surface of the sample and is used here in comparison to the TOF-SIMS data to assess the depth of coverage of the magnesium stearate on the lactose surface.

In addition, the powders were studied using the Zetasizer 3000HS instrument (Malvern Instruments Ltd, UK.) Each sample was tested in cyclohexane, and zeta potential measurements were obtained.

The following powder samples were prepared for testing:

Lactose;

Lactose/Magnesium Stearate 19/1 mixed by Turbula mixer; and

Lactose/Magnesium Stearate 19/1 mixed by Mechanofusion.

### TOF-SIMS

SIMS is a qualitative surface analytical technique that is capable of producing a high-resolution mass spectrum of the outermost 1 nm of a surface.

In brief, the SIMS process involves bombarding the sample surface with a beam of primary ions (for example caesium or gallium). Collision of these ions with atoms and molecules in the surface results in the transfer of energy to them, causing their emission from the surface. The types of particles emitted from the surface include positive and negative ions (termed secondary ions), neutral species and electrons. Only secondary ions are measured in SIMS. Depending on the type of bias applied to the sample, either positive or negative ions are directed towards a mass spectrometer. These ions are then analysed in terms of their mass-to-charge ratio (W [symbol] yielding a positive or negative ion mass spectrum of counts detected versus [symbol] Different fragments will be diagnostic of different components of the surface. TOF-SIMS is an advanced technique that has increased sensitivity (<<parts per million (ppm) sensitivity), mass resolution and mass range compared to conventional SIMS techniques. SIMS operating in static mode was used to determine the chemical composition of the top monolayer of the surface. Under static SIMS conditions, the primary ion dose is limited so that statistically the sample area analysed by the rastered ion beam is exposed to the beam once only, and that the spectrum generated is representative of a pristine surface.

TOF-SIMS analysis of the Turbula mixed sample (Lactose/Magnesium Stearate 19/1 mixed by Turbula) indicated the presence of both lactose and magnesium stearate in both positive and negative mass spectra, as shown in the table below. The presence of lactose in the spectra indicates that the surface coverage of magnesium stearate is incomplete.

TOF-SIMS analysis of the Mechano fusion mixed sample (Lactose/Magnesium Stearate 19/1 co-milled by Mechano fusion) also indicated the presence of both lactose and magnesium stearate in both positive and negative mass spectra (see the following table). The presence of lactose in the spectra indicates that the surface coverage of magnesium stearate is incomplete.

| | Relative Atomic | | |
|---|---|---|---|
| | Percentage | | |
| | Composition (%) | | |
| Sample | C | O | Mg |
| Lactose | | | |
| Measurement 1 | 54.47 | 45.43 | Nd* |
| Measurement 2 | 55.29 | 44.71 | Nd* |
| Mean | 54.9 | 45.1 | <0.1 |
| Lactose/Magnesium Stearate (Turbula) | | | |
| Measurement 1 | 61.23 | 38.00 | 0.44 |
| Measurement 2 | 60.40 | 39.02 | 0.50 |
| Mean | 60.8 | 38.5 | 0.5 |
| Lactose/Magnesium Stearate | | | |
| (Mechanofusion) | | | |
| Measurement 1 | 81.39 | 17.07 | 1.51 |
| Measurement 2 | 80.72 | 17.80 | 1.49 |
| Mean | 81.1 | 17.4 | 1.5 |

*Nd = not detected (<0.1 atomic %)

It is important to note that SIMS spectra are not quantitative and so the intensities of the peaks cannot be taken to reflect the degree of surface coverage.

### XPS

XPS is a surface analytical technique that can quantify the amount of different chemical species in the outermost 10 nm of a surface. In the simplest form of analysis, XPS measures the relative amount of each element present. Quantitative elemental identification can be achieved down to 1 atom in 1000. AU elements present can be detected with the exception of hydrogen. Elemental analysis may be essential in determining the amount of a surface contaminant or to quantify any surface species with a unique elemental type.

XPS analysis of the Lactose/Magnesium Stearate 19/1 sample mixed by Turbula revealed the presence of magnesium on the surface of the lactose indicating the presence of magnesium stearate. Using the percentage presence of magnesium on the surface it is calculated that the magnesium stearate contributes 20% of the overall signal from the outermost 10 nm of the sample surface. Peak fitting the carbon Is envelope enables the identification and quantification of the functionalities present at the surface. The clear increase in C—H/C—C carbon centres at the surface is ascribed to the coverage of magnesium stearate and demonstrates a similar degree of signal intensity to that predicted from the magnesium abundance.

XPS analysis of the Lactose/Magnesium Stearate 19/1 Mechanofusion mixed sample again demonstrates the presence of magnesium stearate on the lactose surface by both the magnesium abundance and the increase in C—C/C—H functionality over that seen on pure lactose. Using the percentage of magnesium in the spectrum the magnesium stearate is calculated to contribute 61.5% of the signal from the outermost 10 nm of the sample surface. An increase of similar magnitude is observed for the C—C/C—H coverage.

The carboxyl functionality present on the surface of the lactose can most likely be attributed to surface contamination, and as such the carboxyl group is not used to assess the degree of magnesium stearate coverage. However for the two mixed samples the extent of carboxyl functionality follows the same trend as for magnesium and the C—C/C—H increases.

The Mechanofusion mixed sample demonstrated significantly increased amounts of magnesium stearate at the surface, over the Turbula mixed sample. These differences could reflect either a thickening of the coverage of magnesium stearate or an increased surface coverage given the incomplete coverage as demonstrated by TOF-SIMS analysis.

| | Area % of C 1s Spectral Envelope | | | |
|---|---|---|---|---|
| Sample | C—C | C—O | O—C—O | O—C═O |
| Lactose | | | | |
| Measurement 1 | 6.4 | 70.9 | 18.0 | 4.7 |
| Measurement 2 | 4.4 | 57.8 | 22.0 | 12.8 |
| Mean | 5.5 | 64.3 | 20.0 | 8.7 |
| Lactose/Magnesium Stearate | | | | |
| (Turbula) | | | | |
| Measurement 1 | 25.8 | 57.5 | 14.7 | 2.1 |
| Measurement 2 | 24.7 | 58.8 | 15.0 | 1.6 |
| Mean | 25.2 | 58.1 | 14.8 | 1.8 |
| Lactose/Magnesium Stearate | | | | |
| (Mechanofusion) | | | | |
| Measurement 1 | 75.7 | 16.1 | 3.9 | 4.3 |
| Measurement 2 | 73.9 | 17.2 | 4.5 | 4.5 |
| Mean | 74.8 | 16.6 | 4.2 | 4.4 |

In conclusion both mixed samples demonstrate an incomplete coverage of magnesium stearate, but with about three times more magnesium stearate present on the Mechanofusion mixed sample than the Turbula sample in the top 10 nm of the surface.

### Zeta Potential

Zetasizer measures the zeta potential. This is a measure of the electric potential on a particle in suspension in the hydrodynamic plane of shear. The results are summarized as follows:

| Sample | Zeta Potential (mV) |
|---|---|
| Lactose | 35.5 |
| Lactose/Magnesium Stearate (19/1) (Turbula) | 4.8 |
| Lactose/Magnesium Stearate | −34.8 |
| (19/1) (Mechanofusion) | |

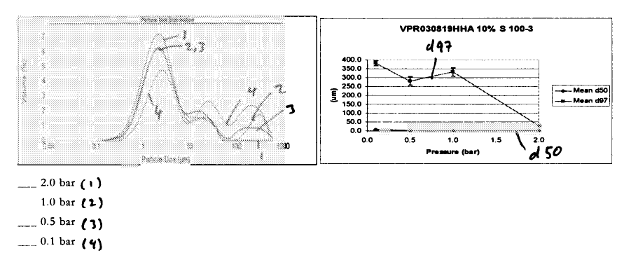

Each result is an average of 10 measurements. The data are presented in FIG. 7. This technique shows a clear difference in the zeta potential measurements, as a function of surface coating process, where the improved covering of magnesium stearate is indicated by an increasingly negative zeta potential.

These results demonstrate that applying the additive material to fine or ultra-fine s carrier or active particles by conventional mixing or blending, for example using a low shear mixer like a Turbula mixer, does not provide the same improvement in powder performance as the use of the co-milling process according to the present invention. The latter processes appear to literally fuse the additive material to the surfaces of the active or carrier particles.

1. A pharmaceutical formulation for delivery in aerosol or spray form, comprising a liquefied propellant gas, a solid particulate pharmaceutically active agent and a dispersing agent, wherein the dispersing agent is fused to the surface of particles of the pharmaceutically active agent.

2. A pharmaceutical formulation as claimed in claim 1, wherein the fused dispersing agent and pharmaceutically active agent form solid composite particles.

3. A pharmaceutical formulation as claimed in claim 1, wherein said particles are suspended or suspendable in the liquefied propellant gas.

4. A pharmaceutical formulation as claimed in claim 1, wherein the particles each comprise a particle of the pharmaceutically active agent at least partially coated with the dispersing agent.

5. A pharmaceutical formulation as claimed in claim 4, wherein the dispersing agent forms an at least partial coating or shell around each of said particles of pharmaceutically active agent; said shell or coating covering at least 50, 70, 80, 90 or 95% of the surface area of the pharmaceutically active agent.

6. A pharmaceutical formulation as claimed in claim 5, wherein the coating or shell of dispersing agent formed around the particles of pharmaceutically active agent has a mean thickness of 1, 0.5 or 0.2 μm or less.

7. A pharmaceutical formulation as claimed in claim 1 substantially free of CFC.

8. A pharmaceutical formulation as claimed in claim 1 that includes less than 3, 2, 1, 0.5, 0.1, 0.01 or 0.001%, or is substantially free of polar co-solvent.

9. A pharmaceutical formulation as claimed in claim 1 that includes less than 3, 2, 1, 0.5, 0.1, 0.01 or 0.001%, or is substantially free of co-solvent.

10. A pharmaceutical formulation as claimed in claim 1 that includes less than 3, 2, 1, 0.5, 0.1, 0.01 or 0.001%, or is substantially free of dissolved dispersing agent.

11. A pharmaceutical formulation as claimed in claim 1 consisting essentially of liquefied propellant gas and composite particles that comprise, pharmaceutically active agent and dispersing agent.

12. A pharmaceutical formulation as claimed in claim 1, wherein the particles of fused pharmaceutically active agent and dispersing agent have an MMAD of between 0.1 and 100 μm.

13. A pharmaceutical formulation as claimed in claim 1, wherein the particles of fused pharmaceutically active agent and dispersing agent have an MMAD of up to about 10 μm.

14. A pharmaceutical formulation as claimed in claim 1, wherein the particles of fused pharmaceutically active agent and dispersing agent have an MMAD more than about 10 μm.

15. A method for preparing a pharmaceutical formulation as claimed in claim 1 comprising fusing the dispersing agent to the surface of particles of a solid particulate pharmaceutically active agent and admixing the solid particulate pharmaceutically active agent and dispersing agent with a liquefied propellant gas.

16. A method as claimed in claim 15, wherein the liquefied propellant gas is admixed with the dispersing agent and particulate pharmaceutically active agent before, during and/or after the dispersing agent is fused to the particulate pharmaceutically active agent.

17. A method as claimed in claim 15, wherein, the dispersing agent is fused to the surface of solid particles of pharmaceutically active agent by a method comprising bringing solid dispersing agent into contact with the particles of pharmaceutically active agent, and applying sufficient mechanical energy to contacting dispersing agent and particles of pharmaceutically active agent to cause fusion between them.

18. A method as claimed in claim 15, wherein the dispersing agent is fused to the surface of the particles of pharmaceutically active agent to form solid composite particles.

19. A method as claimed in claim 18, wherein each composite particle comprises a particle of the pharmaceutically active agent at least partially coated with the dispersing agent, which can be suspended in the liquefied propellant gas.

20. A method as claimed in claim 15, wherein the mechanical energy is applied to a mixture of dispersing agent and active agent particles.

21. A method as claimed in claim 15, wherein the mechanical energy is applied to a dry mixture of dispersing agent and active agent particles.

22. A method as claimed in claim 15, wherein the mechanical energy is provided in the form of simultaneous compression and sheer forces applied to the contacting dispersing agent and active agent particles.

23. A method as claimed in claim 15, wherein the dispersing agent is softer and/or more malleable than the pharmaceutically active agent within the temperature range at which said method is carried out.

24. A method as claimed in claim 15, wherein the dispersing agent is softer and/or more malleable than the pharmaceutically active agent at a temperature in the range of 20-80° C.

25. A pharmaceutical formulation as claimed in claim 1, wherein the dispersing agent is sufficiently soft and malleable, relative to the pharmaceutically active agent, such that it can be deformed, spread across and fused to the surfaces of the pharmaceutically active agent particles by the application of mechanical energy to contacting dispersing agent and particles of pharmaceutically active agent.

26. A method as claimed in claim 20, wherein sufficient mechanical energy is applied to contacting particles of dispersing agent and pharmaceutically active agent to cause the dispersing agent particles to soften and/or distort such that the dispersing agent spreads across to at least partially coat the surfaces of the pharmaceutically active agent particles.

27. A method as claimed in claim 15, wherein the particles of dispersing agent are smaller than the particles of pharmaceutically active agent and each of the composite particles comprises a plurality of dispersing agent particles fused to the surface of a particle of pharmaceutically active agent.

28. A method as claimed in claim 15, wherein MMAD of the particles of active agent is between 0.1 and 100μ and the MMAD of the dispersing agent particles does not exceed 1 μm.

29. A method as claimed in claim 15, wherein the ratio of the MMAD of the dispersing agent particles to the MMAD of the pharmaceutically active agent particles is 1:10 or more.

30. A pharmaceutical formulation, prepared or preparable by a method as claimed in claim 15.

31. A pharmaceutical formulation as claimed in claim 1, wherein the bond between the dispersing agent and the active agent is physical.

32. A pharmaceutical formulation as claimed in claim 31, wherein the bond between the dispersing agent and the active agent involves physisorption of the dispersing agent by the pharmaceutically active agent and/or vice versa.

33. A formulation as claimed in claim 1, wherein the dispersing agent is substantially insoluble in the liquefied propellant gas.

34. A formulation as claimed in claim 1, wherein the dispersing agent reduces the surface free energy of the particles of pharmaceutically active agent.

35. A formulation as claimed in claim 1, wherein the dispersing agent has a molecular weight of at least about 5500 or 6000.

36. A formulation as claimed in claim 1, wherein the composite particles comprise less than 60% by weight dispersing agent.

37. A formulation as claimed in claim 1, wherein the composite particles comprise 40-0.25, 30-0.5, 20-2, 10-2 or 8-3% by weight dispersing agent.

38. A formulation as claimed in claim 1, wherein the dispersing agent is an anti-adherent material.

39. A formulation as claimed in claim 1, wherein the dispersing agent comprises one or more amino acid, amino acid derivative, peptide, peptide derivative, metal stearate, metal palmitate, surface active agent, film forming agent, fatty acid, fatty acid derivative, lipid, lipid like material, lecithin, or phospholipid.

40. A formulation as claimed in claim 1, wherein the dispersing agent comprises magnesium stearate.

41. A formulation as claimed in claim 1, wherein the propellant comprises an HFA, or mixture of HFAs.

42. A formulation as claimed in claim 41, wherein the HFA is HFA-134a or HFA-227.

43. A formulation as claimed in claim 1, wherein the pharmaceutically active agent is one or more of: a steroid, a bronchodilator such as a β₂-agonist, an antimuscarinic or a xanthine; a nitrate; an antihistamine; an antiinflammatory agent; an anticholinergic agent; a leukotriene receptor antagonist; an anti-allergic; an anti-emetic; a hormonal drug (including a hormone analogue); a sympathomimetic drug; an opioid; an analgesic such as a salicylate or a non-steroidal anti-inflammatory drug; an acetylcholinesterase inhibitor; an immunomodulatory; an NMDA receptor antagonist; a hypoglycaemic such as a sulphonylurea; a biguanide or a thiazolidinedione; a narcotic agonist or opiate antidote; a phosphodiesterase inhibitor such as a non-specific phosphodiesterase inhibitor or a phosphodiesterase type 3, type 4 or type 5 inhibitor; an antidepressant such as a tricyclic or tetracyclic antidepressant, a selective serotonin and noradrenaline reuptake inhibitor, a selective serotonin reuptake inhibitor, a selective noradrenaline reuptake inhibitor, a noradrenaline and selective serotonin reuptake inhibitor, a monoamine oxidase inhibitor, a muscarinic antagonist or an azaspirone; a serotonin agonist; a serotonin antagonist; an adrenergic agonist; an adrenergic antagonist; an adrenergic neurone blocker; a benzodiazepine; a mucolytic agent; an antibiotic or antibacterial agent; an anti-fungal drug; an antiviral; a vaccine; an immunoglobulin; a local anaesthetic; an anticonvulsant; an angiotensin converting enzyme inhibitor; an angiotension II receptor blocker; a calcium channel blocker; an alpha-blocker; an antiarrhythmic; an anti-clotting agent; a potassium channel modulator; a cholesterol-lowering drug; a diuretic; a smoking cessation drug; a bisphosphonate; a dopamine agonist; a nucleic-acid medicine; an antipsychotic; and pharmaceutically acceptable salts or derivatives thereof.

44. A medical device for delivering a pharmaceutical formulation in aerosol or spray form, comprising a pharmaceutical formulation as claimed in claim 1.

45. A method of treating a patient comprising administering a therapeutically or prophylactically effective amount of formulation as claimed in claim 1, to said patient.

46. A can, suitable for use in a pMDI device, containing a pharmaceutical formulation as claimed in claim 1.

47. A can as claimed in claim 46, further comprising a metering valve.

48. A method as claimed in claim 15, wherein the ratio of the MMAD of the dispersing agent particles to the MMAD of the pharmaceutically active agent particles is 1:20 or more.

49. A method as claimed in claim 15, wherein the ratio of the MMAD of the dispersing agent particles to the MMAD of the pharmaceutically active agent particles is 1:100 or more.

50. The method of claim 45 further comprising using a device as claimed in claim 44.
